(19) United States
(12) Patent Application Publication
(10) Pub. No.: US 2005/0196753 A1
(43) Pub. Date: Sep. 8, 2005

(54) Human coactivator-associated arginine methyltransferase 1 (hCARM1)

(76) Inventors: Lata Jayaraman, Lawrenceville, NJ (US); Rolf Peter Ryseck, Ewing, NJ (US); Matthew V. Lorenzi, Philadelphia, PA (US); David K. Bol, Gaithersburg, MD (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/449,370

(22) Filed: May 30, 2003

Related U.S. Application Data
(60) Provisional application No. 60/384,348, filed on May 30, 2002.

(51) Int. Cl. C12Q 1/68; G01N 33/53; C07H 21/04; C12N 9/10; C12P 21/02; C12N 5/06

(52) U.S. Cl. 435/6; 435/7.1; 435/69.1; 435/320.1; 435/193; 435/325; 536/23.2

(57) ABSTRACT

Human coactivator-associated arginine methyltransferase 1 (hCARM1) polynucleotides and polypeptides. Also provided are expression vectors, recombinant host cells and processes for producing recombinant host cells, processes for producing said polypeptides, and methods for identifying substances that are capable of interacting with a coactivator-associated arginine methyltransferase 1 molecule.

This application claims the benefit of U.S. Provisional Application No. 60/384,348 filed May 30, 2002, whose contents are incorporated by reference in its entirety.

### BACKGROUND OF THE INVENTION

Nuclear hormone receptors (NHRs) are a related group of hormone-regulated transcriptional activators that include the receptors for steroid and thyroid hormones, retinoic acid, and vitamin D (Tsai et al., Annu. Rev. Biochm. 63, 451 (1994); Beato et al., Cell, 83, 851 (1995); and Mangelsdorf and Evans, ibid., p. 841). Transcriptional activation by NHRs is enhanced by the steroid receptor coactivators (SRC), a family of related 160-kD proteins that includes SRC-1, GRIP1/TIF2 and pCIP/RAC3/ACTR/AIBI/TRAM1 (Torchia et al., Curr. Opin. Cell Biol., 10, 373 (1998). Coactivator-associated arginine methyltransferase 1 (CARM1) was originally identified from a mouse cDNA library (Chen et al., Science, Vol. 284, 2174 (1999)) and functions as a secondary coactivator through its interaction with p160 coactivators. CARM1 binds to the carboxyl-terminal region of p160 coactivators to enhance NHR transcription. Additionally, it has also been shown to methylate histone H3 (Chen et al., supra). Mutations in the methyltransferase domain of CARM1 reduce both enzymatic and coactivator activities, indicating that the methyltransferase activity is closely linked to the function of CARM1 as a coactivator in transcriptional regulation.

Therefore, the development of therapeutics that modulate (i.e., act as antagonists or agonists of CARM1) is important to treat diseases related to transcriptional regulation, such as cancer.

### SUMMARY OF THE INVENTION

The present invention provides human coactivator-associated arginine methyltransferase 1 (hCARM1) polynucleotides and polypeptides.

In one aspect, the invention provides isolated polynucleotides comprising: (a) a nucleotide sequence encoding a coactivator-associated arginine methyltransferase 1 polypeptide wherein the amino acid sequence of the polypeptide and the amino acid sequence of at least one of SEQ ID NO:4 and SEQ ID NO:6 have at least 95% sequence identity; or (b) the complement of the nucleotide sequence, wherein the complement and the nucleotide sequence contain the same number of nucleotides and are 100% complementary. In another aspect, the isolated polynucleotides encode the polypeptide of SEQ ID NO:4 or SEQ ID NO:6. In yet another aspect, the isolated polynucleotides comprise the nucleotide sequence of SEQ ID NO:3 or SEQ ID NO:5.

The invention also provides expression vectors that comprise a polynucleotide of the invention and an expression control sequence operatively linked to the polynucleotide.

The invention further provides processes for producing a recombinant host cell comprising transforming or transfecting a host cell with an expression vector of the invention such that the host cell, under appropriate culture conditions, produces a coactivator-associated arginine methyltransferase 1 polypeptide. The invention also includes recombinant host cells produced by this process.

The invention also includes isolated coactivator-associated arginine methyltransferase 1 polypeptides comprising an amino acid sequence that has at least 95% sequence identity to the amino acid sequence of at least one of SEQ ID NO:4 and SEQ ID NO:6. In one aspect, the polypeptides comprise the amino acid sequence of SEQ ID NO:4 or SEQ ID NO:6.

The invention further includes processes for producing a coactivator-associated arginine methyltransferase 1 polypeptide comprising culturing a recombinant host cell of the invention under conditions sufficient for the production of said polypeptide and recovering the polypeptide.

The invention also includes methods for identifying a substance (e.g., a protein) which is capable of modulating a coactivator-associated arginine methyltransferase 1 molecule or a fragment thereof, said method comprising the steps of: (a) reacting a coactivator-associated arginine methyltransferase 1 polypeptide of the invention with a candidate substance under conditions which permit an interaction between said coactivator-associated arginine methyltransferase 1 polypeptide and said candidate substance; and (b) assaying for one or more of a candidate substance-coactivator-associated arginine methyltransferase 1 polypeptide complex, a free coactivator-associated arginine methyltransferase 1 polypeptide, a non-complexed candidate substance, or activation of the coactivator-associated arginine methyltransferase 1 polypeptide.

### BRIEF DESCRIPTION OF THE FIGURES

FIGS. 1A-F show the polynucleotide sequence of hCARM1-long form (SEQ ID NO:5) aligned with a published sequence (XM_{—}032719) for a clone of hCARM1. The bases of positions 1-11 of the hCARM1-long (SEQ ID NO:5) have been artificially added. The next 710 bases (positions 11-721) were not present in the published sequence. The published sequence contains a sequence error at position 1709 of hCARM1-Long (SEQ ID NO:5) as indicated by the “-” and “*” in FIG. 1E, which results in a change of reading frame.

FIG. 2 shows the efficient methylation of Histone H3 by hCARM1.

FIG. 3 shows the expression levels for hCARM1-long form in various tissue samples, wherein each normal tissue sample is represented by an unpatterned bar and each tumor tissue sample is represented by a patterned bar.

FIG. 4 shows the expression levels for hCARM1-short form in various tissue samples, wherein each normal tissue sample is represented by an unpatterned bar and each tumor tissue sample is represented by a patterned bar.

### DETAILED DESCRIPTION OF THE INVENTION

The invention includes human homologues of CARM1 (“hCARM1”) and the cDNA encoding said hCARM1. The nucleotide sequences of the isolated cDNA are disclosed herein along with the deduced amino acid sequences. The hCARM1s of the invention have homology to known sequences encoding murine CARM1 and other protein arginine methyl transferases (PRMTs).

The hCARM1 of the invention can be produced by: (1) inserting the cDNA of the disclosed hCARM1 into an appropriate expression vector and (2) introducing (e.g., by transfection or injection) the expression vector into an appropriate host(s) (e.g., host cells). This production can further include the steps of (3) growing the host cells in appropriate culture media; and (4) purifying the protein.

The invention therefore provides purified and isolated nucleic acid molecules, preferably DNA molecules, having sequences that encodes for a hCARM1, or an oligonucleotide fragment of the nucleic acid molecule which is unique to the hCARM1 of the invention.

The invention also contemplates a double stranded nucleic acid molecule comprising a nucleic acid molecule of the invention or an oligonucleotide fragment thereof hydrogen bonded to a complementary nucleotide base sequence.

The terms “isolated and purified nucleic acid” and “substantially pure nucleic acid”, e.g., substantially pure DNA, refer to a nucleic acid molecule which is one or both of the following: (1) not immediately contiguous with either one or both of the sequences, e.g., coding sequences, with which it is immediately contiguous (i.e., one at the 5′ end and one at the 3′end) in the naturally occurring genome of the organism from which the nucleic acid is derived; or (2) which is substantially free of a nucleic acid sequence with which it occurs in the organism from which the nucleic acid is derived. The term includes, for example, a recombinant DNA which is incorporated into a vector, e.g., into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment) independent of other DNA sequences. Substantially pure or isolated and purified DNA also includes a recombinant DNA, which is part of a hybrid gene encoding additional hCARM1 sequence.

The invention provides in one embodiment: (a) an isolated and purified nucleic acid molecule comprising a sequence encoding all or a portion of a protein having the amino acid sequence as shown in at least one of SEQ ID NO:4 or 6; (b) nucleic acid sequences complementary to (a); (c) nucleic acid sequences which exhibit at least 80%, more preferably at least 90%, more preferably at least 95%, and most preferably at least 98% sequence identity to (a); or (d) a fragment of (a) or (b) that is at least 18 bases and which will hybridize to (a) or (b) under stringent conditions. In a particular embodiment, the fragment is a sequence encoding a hCARM1 having the amino acid sequence as shown in SEQ ID NO:4 or 6 and sequences having at least 80%, preferably at least 85%, more preferably at least 90%, more preferably at least 95%, more preferably at least 96%, more preferably at least 97%, more preferably at least 98%, and most preferably at least 99% sequence identity thereto.

The degree of homology (percent identity) between a native and a mutant sequence may be determined, for example, by comparing the two sequences using computer programs commonly employed for this purpose. One suitable program is the GAP computer program described by Devereux et al., (1984) Nucl. Acids Res. 12:387. The GAP program utilizes the alignment method of Needleman and Wunsch (1970) J. Mol. Biol. 48:433, as revised by Smith and Waterman (1981) Adv. Appl. Math. 2:482. Briefly, the GAP program defines percent identity as the number of aligned symbols (i.e., nucleotides or amino acids) which are identical, divided by the total number of symbols in the shorter of the two sequences.

As used herein the term “stringent conditions” encompasses conditions known in the art under which a nucleotide sequence will hybridize to an isolated and purified nucleic acid molecule comprising a sequence encoding a protein having the amino acid sequence as shown herein, or to (b) a nucleic acid sequence complementary to (a). Screening polynucleotides under stringent conditions may be carried out according to the method described in Nature, 313:402-404 (1985). Polynucleotide sequences capable of hybridizing under stringent conditions with the polynucleotides of the invention may be, for example, allelic variants of the disclosed DNA sequences, or may be derived from other sources. General techniques of nucleic acid hybridization are disclosed by Sambrook et al., “Molecular Cloning: A Laboratory Manual”, 2nd Ed., Cold Spring Harbor Laboratory, Cold Spring Harbor, N.Y. (1984); and by Haymes et al., “Nucleic Acid Hybridization: A Practical Approach”, IRL Press, Washington, D.C. (1985), which references are incorporated herein by reference.

The invention also provides: (a) a purified and isolated nucleic acid molecule comprising a sequence as shown in SEQ ID NO:3 or 5; (b) nucleic acid sequences complementary to (a); (c) nucleic acid sequences having at least 80%, more preferably at least 90%, more preferably at least 95%, and most preferably at least 98% sequence identity to (a); or (d) a fragment of (a) or (b) that is at least 18 bases and which will hybridize to (a) or (b) under stringent conditions.

The invention also includes nucleic acid and amino acid sequences having one or more structural mutations including replacement, deletion, or insertion mutations from the sequences of SEQ ID NOS:3-6. For example, a signal peptide may be deleted, or conservative amino acid substitutions may be made to generate a protein that is still biologically competent or active.

The invention further contemplates a recombinant molecule comprising a nucleic acid molecule of the invention or an oligonucleotide fragment thereof and an expression control sequence operatively linked to the nucleic acid molecule or oligonucleotide fragment. A transformant host cell including a recombinant molecule of the invention is also provided.

In another aspect, the invention features a cell or purified preparation of cells which include a gene encoding a hCARM1 of the invention, or which otherwise misexpresses a gene encoding a hCARM1 of the invention. The cell preparation can consist of human or non-human cells, e.g., insect cells (e.g., drosophila), rodent cells (e.g., mouse or rat cells), or mammalian cells (e.g., rabbit or pig cells). In preferred embodiments, the cell or cells include a hCARM1 transgene, e.g., a heterologous form of a hCARM1 gene, e.g., a gene derived from humans (in the case of a non-human cell). The hCARM1 transgene can be misexpressed, e.g., overexpressed or underexpressed. In other preferred embodiments, the cell or cells include a gene that misexpresses an endogenous hCARM1 gene, e.g., a gene the expression of which is disrupted, e.g., a knockout. Such cells can serve as a model for studying disorders which are related to mutated or misexpressed hCARM1 alleles for use in drug screening.

Still further, the invention provides plasmids which comprise the nucleic acid molecules of the invention.

The invention also includes a hCARM1 of the invention, or an active part thereof. A biologically competent or active form of the protein or part thereof is also referred to herein as an “active hCARM1 or part thereof”.

The invention further contemplates antibodies having specificity against an epitope of the hCARM1 of the invention or part of the protein. These antibodies may be polyclonal or monoclonal. The antibodies may be labeled with a detectable substance and they may be used, for example, to detect the hCARM1 of the invention in tissue and cells. Additionally, the antibodies of the invention, or portions thereof, may be used to make targeted antibodies that destroy hCARM1 expressing cells (e.g., antibody-toxin fusion proteins or radiolabelled antibodies).

The invention also permits the construction of nucleotide probes that encode part or all of the hCARM1 protein of the invention or a part of the protein. Thus, the invention also relates to a probe comprising a nucleotide sequence coding for a protein, which displays the properties of the hCARM1 of the invention or a peptide unique to the protein. The probe may be labeled, for example, with a detectable (e.g., radioactive) substance and it may be used to select from a mixture of nucleotide sequences a nucleotide sequence coding for a protein which displays the properties of the hCARM1 of the invention.

The invention also provides a transgenic insect or non-human animal (e.g., a rodent, e.g., a mouse or a rat, a rabbit, or a pig) or embryo all of whose germ cells and somatic cells contain a recombinant molecule of the invention, preferably a recombinant molecule comprising a nucleic acid molecule of the invention encoding the hCARM1 of the invention or part thereof. The recombinant molecule may comprise a nucleic acid sequence encoding the hCARM1 of the invention with a structural mutation, or may comprise a nucleic acid sequence encoding the hCARM1 of the invention or part thereof and one or more regulatory elements which differ from the regulatory elements that drive expression of the native protein. In another preferred embodiment, the insect or animal has a hCARM1 gene which is misexpressed or not expressed, e.g., a knockout. Such transgenic animals can serve as a model for studying disorders that are related to mutated or misexpressed hCARM1 of the invention.

The invention still further provides a method for identifying a substance which is capable of binding the hCARM1 of the invention and/or modulating (e.g., activating or inhibiting, preferably inhibiting) one or more activities of a hCARM1 of the invention, comprising reacting the hCARM1 of the invention or part of the protein under conditions which permit the formation of a complex that comprises the substance and the hCARM1 protein or part of the protein, and assaying for substance-hCARM1 complexes, for free substance, for non-complexed hCARM1, or for modulation of the substance (e.g., receptor) that binds to the hCARM1 of the invention.

An embodiment of the invention provides a method for identifying proteins which are capable of binding the hCARM1 protein of the invention, isoforms thereof, or part of the protein, said method comprising reacting the hCARM1 protein of the invention, isoforms thereof, or part of the hCARM1 protein, with at least one protein which potentially is capable of binding to the protein, isoform, or part of the hCARM1 protein, under conditions which permit the formation of hCARM1 protein-protein complexes, and assaying for hCARM1 protein-protein complexes, for free hCARM1 protein, for non-complexed protein, or for activation of the protein. In a preferred embodiment of the method, the protein identified as binding to the hCARM1 protein is a substrate.

The invention also relates to a method for assaying a medium for the presence of an agonist or antagonist of the interaction of the hCARM1 protein and a protein which is capable of binding the hCARM1 (either directly or indirectly) and/or modulating (e.g., activating or inhibitint) the hCARM1, said method comprising providing a known concentration of the hCARM1, reacting the hCARM1 with a protein which is capable of binding the hCARM1 and a suspected agonist or antagonist under conditions which permit the formation of protein-hCARM1 complexes, and assaying for protein-hCARM1 complexes, for free protein, for non-complexed hCARM1, or for modulation (e.g., activation) of the protein.

Also included within the scope of the invention is a composition which includes the hCARM1 of the invention, a fragment thereof (or a nucleic acid encoding said hCARM1 or fragment thereof) and one or more additional components, e.g., a carrier, diluent, or solvent. The additional component can be one which renders the composition useful for in vitro, in vivo, pharmaceutical, or veterinary use.

In another aspect, the invention relates to a method of treating a mammal, e.g., a human, at risk for a disorder, e.g., a disorder characterized by aberrant or unwanted level or biological activity of the hCARM1 of the invention, or characterized by an aberrant or unwanted level of a ligand that specifically binds the hCARM1 of the invention. For example, the hCARM1 of the invention may be useful to leach out or block a ligand that is found to bind to the hCARM1 of the invention.

The invention provides the identification of new molecules (e.g., a human homologue) homologous to the hCARM1 provided herein, and methods of screening for molecules that modulate the biological activities of the hCARM1 disclosed herein. In addition, the invention provides methods of using the cDNA, the hCARM1 protein, the monoclonal antibody specific for the hCARM1, and a ligand for the hCARM1.

A complete full length hCARM1 cDNA sequence was electronically assembled using the RefSeq entry XM_{—}032719 encoding a partial clone as a starting sequence and public expressed sequence tag (“EST”) sequences as a source for clone and sequence information. The resulting “raw” sequence was compared to the human genomic database and several genomic clones (AC007565, AC011442) were identified. The exon sequence information was used to clean up the initially assembled “raw sequence.” The resulting corrected amino acid sequence was compared to the peptide encoded by the murine CARM1 (RefSeq NM_{—}021531) to ensure reliability of the hypothetical human product.

In order to clone the human coding region of CARM1 the following oligonucleotides were designed:

| | |
|---|---|
| CARM1-PCR3: | |
| CACCGAATTCGCCGGATCTAAGATGGCAGCGGCGG | (SEQ ID NO:1) |
| CARM1-PCR5STOP: | |
| CTAGCTCCCGTAGTGCATGGTGTTGGTCGG. | (SEQ ID NO:2) |

PCR conditions utilized were: 95° C. denaturing temperature for 30 minutes, annealing using a temperature gradient thermocycler (Eppendorf Mastercycler) with a range of 50° C. to 70° C. for one hour and 30 minutes, followed by synthesis at 72° C. for two hours and 30 minutes). A mixture of cDNAs from different sources (cancer cell lines, human spleen, brain, placenta, liver) was used as a template and Pfu polymerase (Stratagene) as the enzyme in the presence of 10% DMSO, 250 μM dNTPs, 1×Pfu reaction buffer. The resulting PCR product was gel purified and cloned using the pENTR Directional TOPO Cloning Kit (Invitrogen), and several independent clones were sequenced.

Two cDNA products were identified, and were designated as hCARM1-long form (also referred to herein as “hCARM1-long”) and hCARM1-short form (also referred to herein as “hCARM1-short”), wherein hCARM1-long encodes a protein having an additional 23 amino acids compared to hCARM1-short. The additional 23 amino acids of hCARM1-long occur at positions 539 to 561 of SEQ ID NO:6. The polynucleotide and polypeptide sequences for the two identified hCARM1 clones are:

hCARM1-Short - DNA Sequence

(SEQ ID NO:3)

CACCGAATTCGCCGGATCTAAGATGGCAGCGGCGGCGGCGGCGGTGGGG
CCGGGCGCGGGCGGCGCGGGGTCGGCGGTCCCGGGCGGCGCGGGGCCCT
GCGCTACCGTGTCGGTGTTCCCCGGCGCCCGCCTCCTCACCATCGGCGAC
GCGAACGGCGAGATCCAGCGGCACGCGGAGCAGCAGGCGCTGCGCCTCGA
GGTGCGCGCCGGCCCGGACTCGGCGGGCATCGCCCTCTACAGCCATGAAG
ATGTGTGTGTCTTTAAGTGCTCAGTGTCCCGAGAGACAGAGTGCAGCCGT
GTGGGCAAGCAGTCCTTCATCATCACCCTGGGCTGCAACAGCGTCCTCAT
CCAGTTCGCCACACCCAACGATTTCTGTTCCTTCTACAACATCCTGAAAA
CCTGCCGGGGCCACACCCTGGAGCGGTCTGTGTTCAGCGAGCGGACGGAG
GAGTCTTCTGCCGTGCAGTACTTCCAGTTTTATGGCTACCTGTCCCAGCA
GCAGAACATGATGCAGGACTACGTGCGGACAGGCACCTACCAGCGCGCCA
TCCTGCAAAACCACACCGACTTCAAGGACAAGATCGTTCTTGATGTTGGC
TGTGGCTCTGGGATCCTGTCGTTTTTTGCCGCCCAAGCTGGAGCACGGAA
AATCTACGCGGTGGAGGCCAGCACCATGGCCCAGCACGCTGAGGTCTTGG
TGAAGAGTAACAACCTGACGGACCGCATCGTGGTCATCCCGGGCAAGGTG
GAGGAGGTGTCACTCCCCGAGCAGGTGGACATCATCATCTCGGAGCCCAT
GGGCTACATGCTCTTCAACGAGCGCATGCTGGAGAGCTACCTCCACGCCA
AGAAGTACCTGAAGCCCAGCGGAAACATGTTTCCTACCATTGGTGACGTC
CACCTTGCACCCTTCACGGATGAACAGCTCTACATGGAGCAGTTCACCAA
GGCCAACTTCTGGTACCAGCCATCTTTCCATGGAGTGGACCTGTCGGCC
CTCCGAGGTGCCGCGGTGGATGAGTATTTCCGGCAGCCTGTGGTGGACAC
ATTTGACATCCGGATCCTGATGGCCAAGTCTGTCAAGTACACGGTGAACT
TCTTAGAAGCCAAAGAAGGAGATTTGCACAGGATAGAAATCCCATTCAAA
TTCCACATGCTGCATTCAGGGCTGGTCCACGGCCTGGCTTTCTGGTTTGA
CGTTGCTTTCATCGGCTCCATAATGACCGTGTGGCTGTCCACAGCCCCGA
CAGAGCCCCTGACCCACTGGTACCAGGTGCGGTGCCTGTTCCAGTCACCA
CTGTTCGCCAAGGCAGGGGACACGCTCTCAGGGACATGTCTGCTTATTGC
CAACAAAAGACAGAGCTACGACATCAGTATTGTGGCCCAGGTGGACCAGA
CCGGCTCCAAGTCCAGTAACCTCCTGGATCTGAAAAACCCCTTCTTTAGA
TACACGGGCACAACGCCCTCACCCCCACCCGGCTCCCACTACACATCTCC
CTCGGAAAACATGTGGAACACGGGCAGCACCTACAACCTCAGCAGCGGGA
TGGCCGTGGCAGGGATGCCGACCGCCTATGACTTGAGCAGTGTTATTGCC
AGTGGCTCCAGCGTGGGCCACAACAACCTGATTCCTTTAGGGTCCTCCGG
CGCCCAGGGCAGTGGTGGTGGCAGCACGAGTGCCCACTATGCAGTCAACA
GCCAGTTCACCATGGGCGGCCCCGCCATCTCCATGGCGTCGCCCATGTCC
ATCCCGACCAACACCATGCACTACGGGAGCTAG

hCARM1-Short - Peptide Sequence

(SEQ ID NO:4)

MAAAAAAVGPGAGGAGSAVPGGAGPCATVSVFPGARLLTIGDANGEIQRH
AEQQALRLEVRAGPDSAGIALYSHEDVCVFKCSVSRETECSRVGKQSFII
TLGCNSVLIQFATPNDFCSFYNILKTCRGHTLERSVFSERTEESSAVQYF
QFYGYLSQQQNMMQDYVRTGTYQRAILQNHTDFKDKIVLDVGCGSGILSF
FAAQAGARKIYAVEASTMAQHAEVLVKSNNLTDRIVVIPGKVEEVSLPEQ
VDIIISEPMGYMLFNERMLESYLHAKKYLKPSGNMFPTIGDVHLAPFTDE
QLYMEQFTKANFWYQPSFHGVDLSALRGAAVDEYFRQPVVDTFDIRILMA
KSVKYTVNFLEAKEGDLHRIEIPFKFHMLHSGLVHGLAFWFDVAFIGSIM
TVWLSTAPTEPLTHWYQVRCLFQSPLFAKAGDTLSGTCLLIANKRQSYDI
SIVAQVDQTGSKSSNLLDLKNPFFRYTGTTPSPPPGSHYTSPSENMWNTG
STYNLSSGMAVAGMPTAYDLSSVIASGSSVGHNNLIPLGSSGAQGSGGGS
TSAHYAVNSQFTMGGPAISMASPMSIPTNTMHYGS.

hCARM1-Long - DNA Sequence

(SEQ ID NO:5)

CACCGAATTCGCCGGATCTAAGATGGCAGCGGCGGCGGCGGCGGTGGGG
CCGGGCGCGGGCGGCGCGGGGTCGGCGGTCCCGGGCGGCGCGGGGCCCT
GCGCTACCGTGTCGGTGTTCCCCGGCGCCCGCCTCCTCACCATCGGCGAC
GCGAACGGCGAGATCCAGCGGCACGCGGAGCAGCAGGCGCTGCGCCTCGA
GGTGCGCGCCGGCCCGGACTCGGCGGGCATCGCCCTCTACAGCCATGAAG
ATGTGTGTGTCTTTAAGTGCTCAGTGTCCCGAGAGACAGAGTGCAGCCGT
GTGGGCAAGCAGTCCTTCATCATCACCCTGGGCTGCAACAGCGTCCTCAT
CCAGTTCGCCACACCCAACGATTTCTGTTCCTTCTACAACATCCTGAAAA
CCTGCCGGGGCCACACCCTGGAGCGGTCTGTGTTCAGCGAGCGGACGGAG
GAGTCTTCTGCCGTGCAGTACTTCCAGTTTTATGGCTACCTGTCCCAGCA
GCAGAACATGATGCAGGACTACGTGCGGACAGGCACCTACCAGCGCGCCA
TCCTGCAAAACCACACCGACTTCAAGGACAAGATCGTTCTTGATGTTGGC
TGTGGCTCTGGGATCCTGTCGTTTTTTGCCGCCCAAGCTGGAGCACGGAA
AATCTACGCGGTGGAGGCCAGCACCATGGCCCAGCACGCTGAGGTCTTGG
TGAAGAGTAACAACCTGACGGACCGCATCGTGGTCATCCCGGGCAAGGTG
GAGGAGGTGTCACTCCCCGAGCAGGTGGACATCATCATCTCGGAGCCCAT
GGGCTACATGCTCTTCAACGAGCGCATGCTGGAGAGCTACCTCCACGCCA
AGAAGTACCTGAAGCCCAGCGGAAACATGTTTCCTACCATTGGTGACGTC
CACCTTGCACCCTTCACGGATGAACAGCTCTACATGGAGCAGTTCACCAA
GGCCAACTTCTGGTACCAGCCATCTTTCCATGGAGTGGACCTGTCGGCCC
TCCGAGGTGCCGCGGTGGATGAGTATTTCCGGCAGCCTGTGGTGGACACA
TTTGACATCCGGATCCTGATGGCCAAGTCTGTCAAGTACACGGTGAACTT
CTTAGAAGCCAAAGAAGGAGATTTGCACAGGATAGAAATCCCATTCAAAT
TCCACATGCTGCATTCAGGGCTGGTCCACGGCCTGGCTTTCTGGTTTGAC
GTTGCTTTCATCGGCTCCATAATGACCGTGTGGCTGTCCACAGCCCCGAC
AGAGCCCCTGACCCACTGGTACCAGGTGCGGTGCCTGTTCCAGTCACCAC
TGTTCGCCAAGGCAGGGGACACGCTCTCAGGGACATGTCTGCTTATTGCC
AACAAAAGACAGAGCTACGACATCAGTATTGTGGCCCAGGTGGACCAGAC
CGGCTCCAAGTCCAGTAACCTCCTGGATCTGAAAAACCCCTTCTTTAGAT
ACACGGGCACAACGCCCTCACCCCCACCCGGCTCCCACTACACATCTCCC
TCGGAAAACATGTGGAACACGGGCAGCACCTACAACCTCAGCAGCGGGAT
GGCCGTGGCAGGGATGCCGACCGCCTATGACTTGAGCAGTGTTATTGCCA
GTGGCTCCAGCGTGGGCCACAACAACCTGATTCCTTTAGCCAACACGGGG
ATTGTCAATCACACCCACTCCCGGATGGGCTCCATAATGAGCACGGGGAT
TGTCCAAGGGTCCTCCGGCGCCCAGGGCAGTGGTGGTGGCAGCACGAGTG
CCCACTATGCAGTCAACAGCCAGTTCACCATGGGCGGCCCCGCCATCTCC
ATGGCGTCGCCCATGTCCATCCCGACCAACACCATGCACTACGGGAGCTA
G

hCARM1-Long - Peptide Sequence

(SEQ ID NO:6)

MAAAAAAVGPGAGGAGSAVPGGAGPCATVSVFPGARLLTIGDANGE
IQRHAEQQALRLEVRAGPDSAGIALYSHEDVCVFKCSVSRETECSRVG
KQSFIITLGCNSVLIQFATPNDFCSFYNILKTCRGHTLERSVFSERTEES
SAVQYFQFYGYLSQQQNMMQDYVRTGTYQRAILQNHTDFKDKIVLDV
GCGSGILSFFAAQAGARKIYAVEASTMAQHAEVLVKSNNLTDRIVVIP
GKVEEVSLPEQVDIIISEPMGYMLFNERMLESYLHAKKYLKPSGNMFP
TIGDVHLAPFTDEQLYMEQFTKANFWYQPSFHGVDLSALRGAAVDE
YFRQPVVDTFDIRILMAKSVKYTVNFLEAKEGDLHRIEIPFKFHMLHS
GLVHGLAFWFDVAFIGSIMTVWLSTAPTEPLTHWYQVRCLFQSPLFA
KAGDTLSGTCLLIANKRQSYDISIVAQVDQTGSKSSNLLDLKNPFFRYT
GTTPSPPPGSHYTSPSENMWNTGSTYNLSSGMAVAGMPTAYDLSSVI
ASGSSVGHNNLIPLANTGIVNHTHSRMGSIMSTGIVQGSSGAQGSGGGS
TSAHYAVNSQFTMGGPAISMASPMSIPTNTMHYGS.

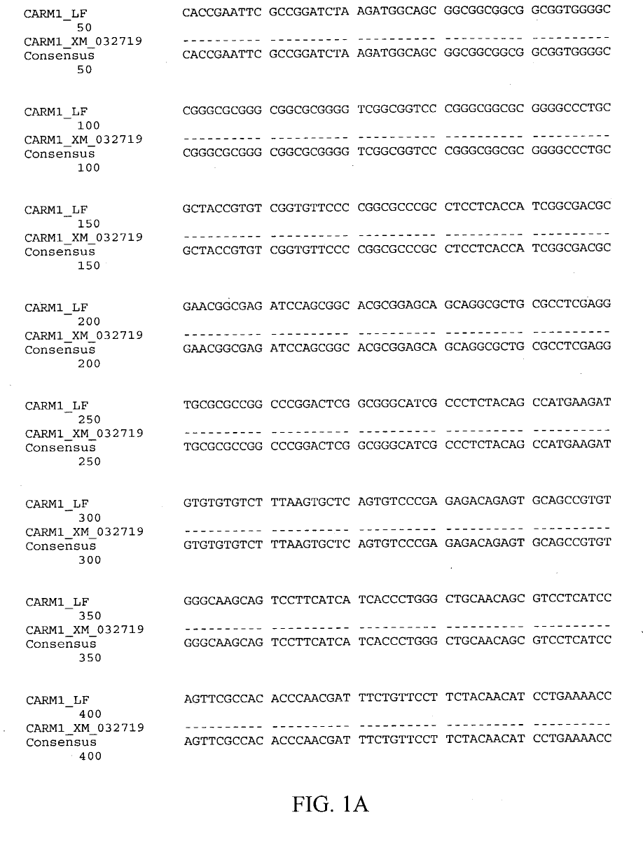

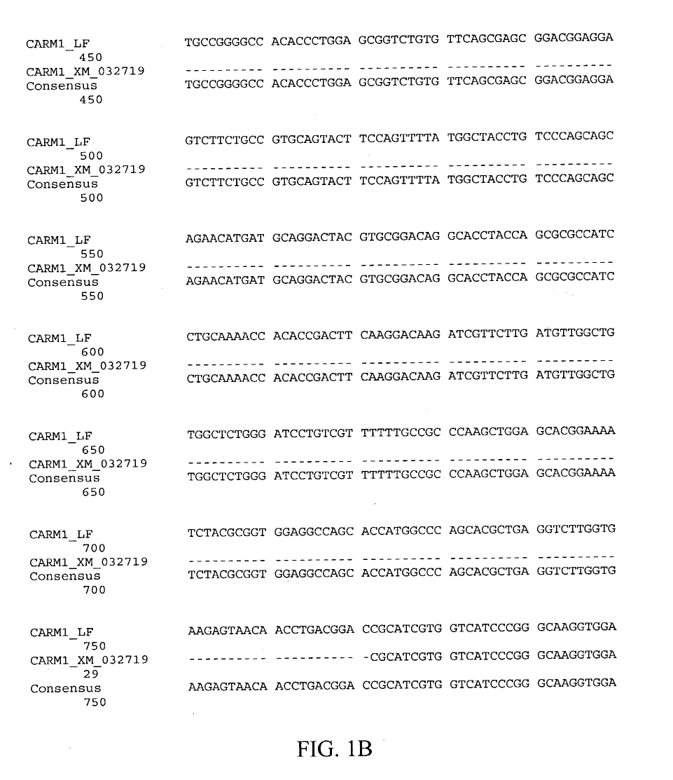

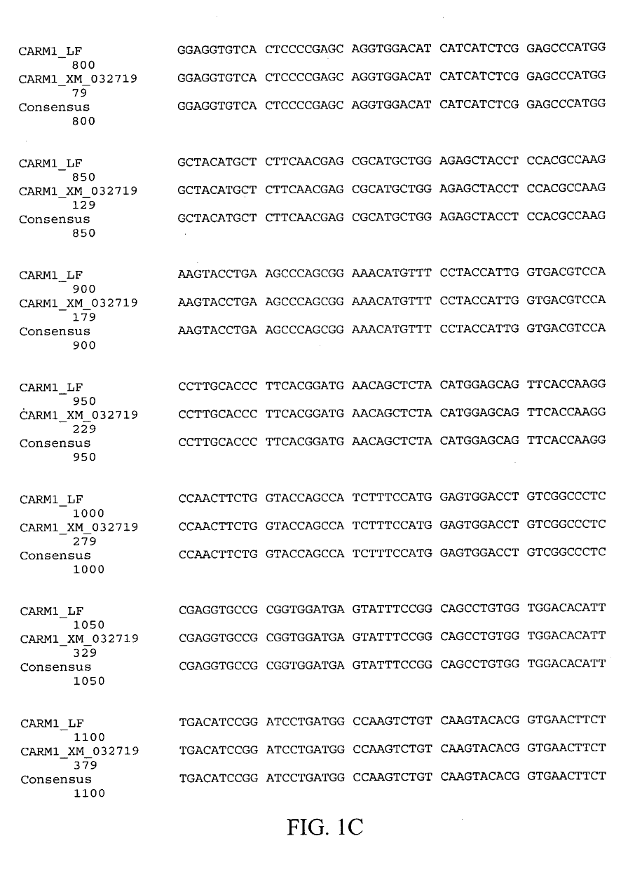

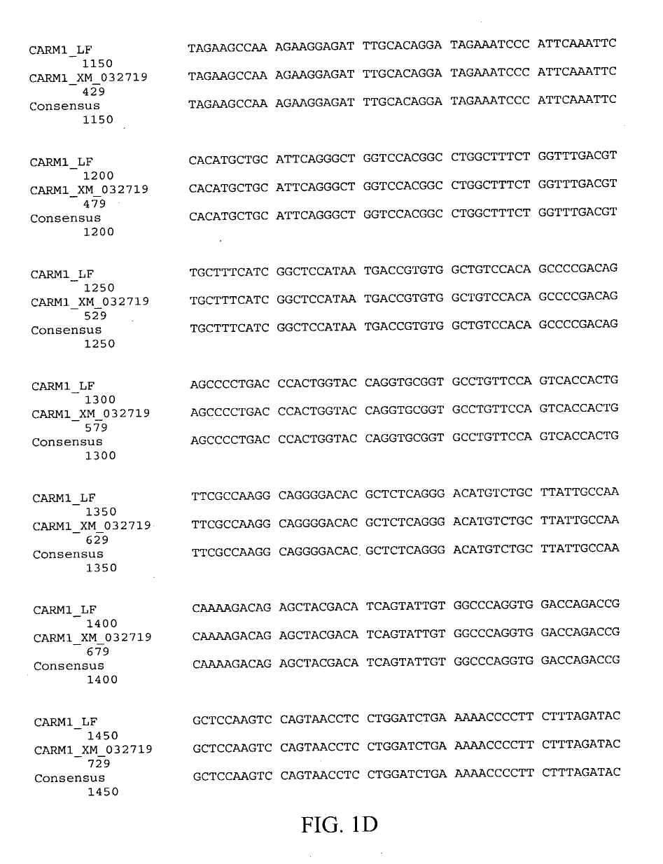

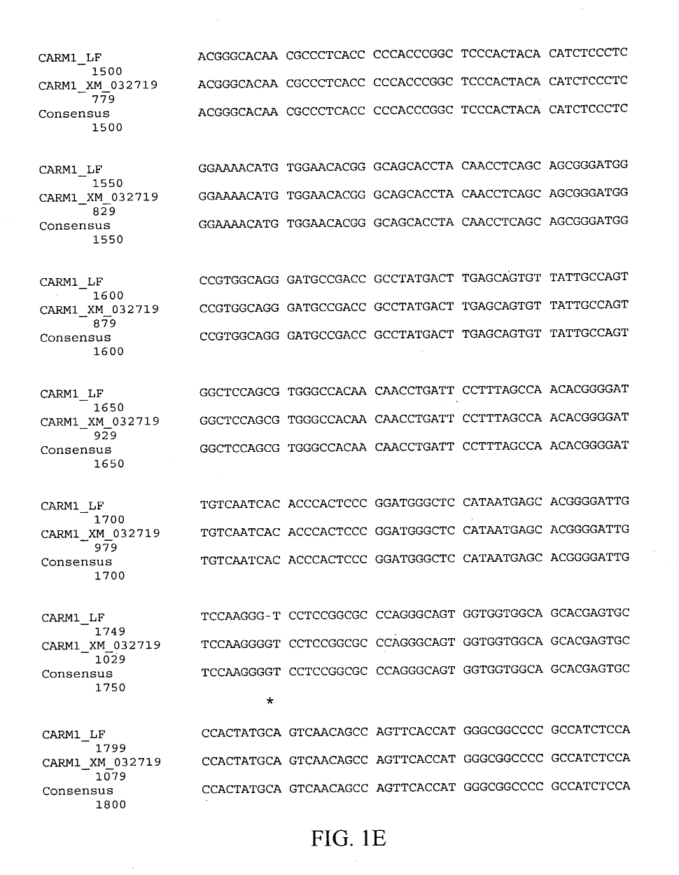

Alignment of hCARM1-Long (SEQ ID NO:5) with the published partial sequence for hCARM1 (XM_{—}032719) indicates that there is a sequence error at position 1709 of the published sequence (FIGS. 1A-1F). This sequence error results in a change of reading frame and hence a different encoded peptide from that of SEQ ID NO:6.

In order to compare the expression levels of CARM1 in human tumors and normal tissues, two distinct approaches were undertaken. First, human CARM1 (hCARM1) message levels in a wide variety of well-characterized tumor cell-lines were analyzed using Taqman. The results indicated that hCARM1 was significantly up-regulated in a variety of tumor derived cell-lines and tissue samples from patients. Second, CARM1 protein levels in multiple tumor biopsy samples and their adjacent normal tissue counterparts were stained with an anti-CARM1 specific antibody. The results showed elevated CARM1 levels in many tumor derived tissues but not in the corresponding normal tissue.

The invention relates to nucleic acid sequences or a fragment thereof (referred to herein as a “polynucleotide”) of the hCARM1 as shown above (SEQ ID NO:3 and SEQ ID NO:5), as well as to the amino acid sequences of hCARM1 (SEQ ID NO:4 and SEQ ID NO;6), and biologically active portions thereof.

The invention further relates to variants of the hereinabove described nucleic acid sequences which encode for fragments, analogs, and derivatives of the polypeptides having the deduced amino acid sequences of SEQ ID NO:4 and SEQ ID NO:6. The variants of these nucleic acid sequences may be naturally occurring variants of the nucleic acid sequences or non-naturally occurring variants of the nucleic acid sequence.

Thus, the invention includes polynucleotides encoding the same mature polypeptides as shown in SEQ ID NO:4 and SEQ ID NO:6, as well as variants of such polynucleotides which variants encode for a fragment, derivative, or analog of the polypeptides of SEQ ID NO:4 and SEQ ID NO:6. Such nucleotide variants include deletion variants, substitution variants, and addition or insertion (splice) variants.

The term “gene” means the segment of DNA involved in producing a polypeptide chain; it includes regions preceding and following the coding region (leader and trailer) as well as intervening sequences (introns) between individual coding segments (exons).

Fragments of the full-length gene of the invention may be used as hybridization probes for a cDNA library to isolate the full-length gene and to isolate other genes which have a high sequence similarity to a gene of the invention or similar biological activity. Probes of this type preferably have at least between 20 and 30 bases, and may contain, for example, 50 or more bases. The probes may also be used to identify a cDNA clone corresponding to a full-length transcript and a genomic clone or clones that contain the complete gene of the invention including regulatory and promoter regions, exons, and introns.

The invention further relates to polynucleotides that hybridize to the polynucleotide sequences disclosed herein, if there is at least 80%, preferably at least 90%, and more preferably at least 95% identity between the sequences. The invention particularly relates to polynucleotides which hybridize under stringent conditions to the polynucleotides described herein.

Alternatively the polynucleotide may have at least 20 bases, preferably at least 30 bases, and more preferably at least 50 bases which hybridize to a polynucleotide of the invention and which has an identity thereto, as hereinabove described, and which may or may not retain activity. For example, such polynucleotides may be employed as probes for the polynucleotide of SEQ ID NO:1, for example for recovery of the polynucleotide or as a diagnostic probe or as a PCR primer.

Thus the invention is directed to polynucleotides having at least 80% identity, preferably at least 90% and more preferably at least 95% identity to a polynucleotide of the invention, including polynucleotides encoding the polypeptides of SEQ ID NO:4 and SEQ ID NO:6, as well as fragments thereof, which fragments have at least 20 or 30 bases, and preferably at least 50 bases, and to polypeptides encoded by such polynucleotides.

The invention further relates to a coactivator-associated arginine methyltransferase 1 molecule polypeptide, hCARM1, which has the deduced amino acid sequences as shown in SEQ ID NO:4 and SEQ ID NO:6, as well as fragments, analogs, and derivatives of such polypeptide.

Analogs of the hCARM1 of the invention are also within the scope of the invention. Analogs can differ from the naturally occurring hCARM1 of the invention in amino acid sequence or in ways that do not involve sequence, or both. Non-sequence modifications include in vivo or in vitro chemical derivitization. Non-sequence modifications include changes in acetylation, methylation, phosphorylation, carboxylation, or glycosylation.

Preferred analogs include the hCARM1 of the invention (or biologically active fragments thereof) whose sequences differ from the wild-type sequences by one or more conservative amino acid substitutions or by one or more non-conservative amino acid substitutions, deletions, or insertions which do not abolish the biological activity of the hCARM1. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions can be taken from Table 1 below.

TABLE 1
Conservative Amino Acid Replacements

| For Amino Acid | Code | Replace with any of: |
|---|---|---|
| Alanine | A | D-Ala, Gly, beta-Ala, L-Cys, D-Cys |
| Arginine | R | D-Arg, Lys, D-Lys, homo-Arg, D-homo-Arg, Met, Ile, D- |
| | | Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, β-Ala, Acp |
| Isoleucine | I | D-Ile, Val, D-Val, Leu, D-Leu, Met, D-Met |
| Leucine | L | D-Leu, Val, D-Val, Met, D-Met |
| Lysine | K | D-Lys, Arg, D-Arg, homo-Arg, D-homo-Arg, Met, D-Met, |
| | | Ile, D-Ile, Orn, D-Orn |
| Methionine | M | D-Met, S-Me-Cys, Ile, D-Ile, Leu, D-Leu, Val, D-Val |
| Phenylalanine | F | D-Phe, Tyr, D-Thr, L-Dopa, His, D-His, Trp, D-Trp, Trans- |
| | | 3,4, or 5-phenylproline, cis-3,4, or 5-phenylproline |
| Proline | P | D-Pro, L-1-thioazolidine-4-carboxylic acid, D- or L-1- |
| | | oxazolidine-4-carboxylic acid |
| Serine | S | D-Ser, Thr, D-Thr, allo-Thr, Met, D-Met, Met(O), D- |
| | | Met(O), L-Cys, D-Cys |
| Threonine | T | D-Thr, Ser, D-Ser, allo-Thr, Met, D-Met, Met(O), D- |
| | | Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Other analogs within the invention are those with modifications which increase protein or peptide stability; such analogs may contain, for example, one or more non-peptide bonds (which replace the peptide bonds) in the protein or peptide sequence. Also included are analogs that include residues other than naturally occurring L-amino acids, e.g., D-amino acids or non-naturally occurring or synthetic amino acids, e.g., β or γ amino acids.

In terms of general utility of the hCARM1 of the invention, gene expression profiling of hCARM1 suggests it is important in human cancers. Such a cancer may include, but is not limited to, adenocarcinoma, leukemia, lymphoma, melanoma, myeloma, sarcoma, teratocarcinoma and, in particular, cancers of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostrate, salivary glands, skin, spleen, testis, thymus, thyroid, and uterus. As such, any of the proteins, antagonists, antibodies, agonists, complementary sequences, or vectors of the invention may be administered to a subject to treat or prevent a cancer.

Gene constructs of the invention can also be used as part of a gene therapy protocol to deliver nucleic acids encoding the hCARM1 of the invention, or an agonist or antagonist form of a hCARM1 protein or peptide. The invention features expression vectors for in vivo transfection and expression of a hCARM1. Expression constructs of the hCARM1 of the invention may be administered in any biologically effective carrier, e.g., any formulation or composition capable of effectively delivering the hCARM1 gene to cells in vivo. Approaches include insertion of the subject gene in viral vectors including recombinant retroviruses, adenoviruses, adeno-associated viruses, and herpes simplex virus-1, or recombinant bacterial or eukaryotic plasmids. Viral vectors transfect cells directly; an advantage of infection of cells with a viral vector is that a large proportion of the targeted cells can receive the nucleic acid. Several viral delivery systems are known in the art and can be utilized by one practicing the invention.

In addition to viral transfer methods, non-viral methods may also be employed to cause expression of the hCARM1 in the tissue of an insect or animal. Most non-viral methods of gene transfer rely on normal mechanisms used by mammalian cells for the uptake and intracellular transport of macromolecules. Exemplary gene delivery systems of this type include liposomal derived systems, poly-lysine conjugates, and artificial viral envelopes. DNA of the invention may also be introduced to cell(s) by direct injection of the gene construct or electroporation.

In clinical settings, the gene delivery systems for the therapeutic hCARM1 gene (or homologue thereof identified using all or a portion of the gene disclosed herein) can be introduced into a patient by any of a number of methods, each of which is known in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof.

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells which produce the gene delivery system.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention.

Another aspect of the invention relates to the use of an isolated nucleic acid in “antisense” therapy. As used herein, “antisense” therapy refers to administration or in situ generation of oligonucleotides or their derivatives which specifically hybridize under cellular conditions with the cellular mRNA and/or genomic DNA encoding the hCARM1 of the invention so as to inhibit expression of the encoded protein, e.g., by inhibiting transcription and/or translation. In general, “antisense” therapy refers to the range of techniques generally employed in the art, and includes any therapy which relies on specific binding to oligonucleotide sequences.

Fragments of the hCARM1 of the invention are also within the scope of the invention. Fragments of the protein can be produced in several ways, e.g., recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid which encodes the polypeptide. Digestion with “end-nibbling” endonucleases can thus generate DNAs which encode an array of fragments. DNAs which encode fragments of the hCARM1 protein can also be generated by random shearing, restriction digestion, or a combination of the above-discussed methods.

Fragments can also be chemically synthesized using techniques known in the art such as conventional Merrifield solid phase f-Moc or t-Boc chemistry.

Amino acid sequence variants of the hCARM1 protein can be prepared by random mutagenesis of DNA which encodes a protein or a particular domain or region of the protein. Useful methods are known in the art, e.g., PCR mutagenesis and saturation mutagenesis. A library of random amino acid sequence variants can also be generated by the synthesis of a set of degenerate oligonucleotides sequences, a process known and practiced by those skilled in the art.

Non-random or directed mutagenesis techniques can be used to provide specific sequences or mutations in specific regions. These techniques can be used to create variants, which include, e.g., deletions, insertions, or substitutions of residues of the amino acid sequences of the hCARM1 protein provided herein. The sites for mutation can be modified individually or in series, e.g., by (1) substituting first with conserved amino acids then with more radical choices depending upon results achieved; (2) deleting the target residue; or (3) inserting residues of the same or a different class (e.g., hydrophobic or hydrophilic) adjacent to the located site, or a combination of options (1)-(3). Alanine scanning mutagenesis is a useful method for identification of certain functional residues or regions of a desired protein that are preferred locations or domains for mutagenesis. Oligonucleotide-mediated mutagenesis, cassette mutagenesis, and combinatorial mutagenesis are useful methods known to those skilled in the art for preparing substitution, deletion, and insertion variants of DNA.

The invention also relates to methods of screening. Various techniques are known in the art for screening generated mutant gene products. Techniques for screening large gene libraries often include cloning the gene library into replicable expression vectors, transforming appropriate cells with the resulting library of vectors, and expressing the genes under conditions in which detection of a desired activity, e.g., in this case binding of the hCARM1 of the invention to an interacting protein (e.g., substrate). Techniques known in the art are amenable to high through-put analysis for screening large numbers of sequences created, e.g., by random mutagenesis techniques.

Two hybrid assays can be used to identify modulators of the interaction of a protein and hCARM1. These modulators may include agonists or antagonists. In one approach to screening assays, the candidate protein or peptides are displayed on the surface of a cell or viral particle, and the ability of particular cells or viral particles to bind an appropriate receptor protein via the displayed product is detected in a “panning assay.” In a similar fashion, a detectably labeled ligand can be used to score for potentially functional peptide homologues. Fluorescently labeled ligands, e.g., receptors, can be used to detect homologue which retain ligand-binding activity. The use of fluorescently labeled ligand allows cells to be visually inspected and separated under fluorescence microscope or to be separated by a fluorescence-activated cell sorter.

High through-put assays can be followed by secondary screens in order to identify further biological activities which will, for example, allow one skilled in the art to differentiate agonists from antagonists. The type of secondary screen used will depend on the desired activity that needs to be tested. For example, an assay can be developed in which the ability to modulate (e.g., inhibit) an interaction between an interacting protein and the hCARM1 of the invention can be used to identify antagonists from a group of peptide fragments isolated through one of the primary screens. Therefore, methods for generating fragments and analogs and testing them for activity are known in the art. Once a sequence of interest is identified, it is routine for one skilled in the art to obtain agonistic or antagonistic analogs, fragments, and/or ligands.

Drug screening assays are also provided in the invention. By producing purified and recombinant hCARM1 of the invention, or fragments thereof, one skilled in the art can use these to screen for drugs which are either agonists or antagonists of the normal cellular function or their role in cellular signaling. In one embodiment, the assay evaluates the ability of a compound to modulate binding between an interacting protein and the hCARM1 of the invention. The term “modulating” encompasses enhancement, diminishment, activation, or inactivation of the receptor for hCARM1. Assays useful to identify a modulator to the hCARM1 of the invention are encompassed herein. A variety of assay formats will suffice and are known by those skilled in the art.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as primary screens in that they can be generated to permit rapid development and relatively easy detection of an alteration in a molecular target which is mediated by a test compound.

Also within the scope of the invention is a process for modulating the activity of hCARM1, either directly or through a protein that interacts with the hCARM1 disclosed herein. The term “modulating” encompasses enhancement, diminishment, activation, or inactivation of the activity of the hCARM1 disclosed herein. Also encompassed herein are molecules (e.g., proteins) that bind or otherwise interact with the hCARM1 disclosed herein (e.g., antibodies specific for the hCARM1 of the invention). These molecules are useful in modulating the activity of the hCARM1 and in treating hCARM1-associated disorders. “hCARM1-associated disorders” refers to any disorder or disease state in which the hCARM1 protein plays a regulatory role in the metabolic pathway of that disorder or disease. Such disorders or diseases may include cancer, as described above. As used herein the term “treating” refers to the alleviation of symptoms of a particular disorder in a patient, the improvement of an ascertainable measurement associated with a particular disorder, or the prevention of a particular immune, inflammatory, or cellular response (such as transplant rejection).

The invention also includes antibodies specifically reactive with the hCARM1 of the invention, or a portion thereof. Anti-protein/anti-peptide antisera or monoclonal antibodies can be made by standard known procedures. A mammal such as a mouse, a hamster, or rabbit can be immunized with an immunogenic form of the peptide. Techniques for conferring immunogenicity on a protein or peptide include conjugation to carriers or other techniques known in the art. An immunogenic portion of the hCARM1 of the invention can be administered in the presence of adjuvant. The progress of immunization can be monitored by detection of antibody titers in plasma or serum.

The term “antibody” as used herein is intended to include fragments thereof which are also specifically reactive with the hCARM1 of the invention. Antibodies can be fragmented using conventional techniques and the fragments screened for utility in the same manner as whole antibodies. For example, F(ab′)2 fragments can be generated by treating antibody with pepsin. The resulting F(ab′)2 fragment can be treated to reduce disulfide bridges to produce Fab′ fragments. The antibody of the invention is further intended to include chimeric and humanized molecules that recognize and bind to the hCARM1 of the invention.

Both monoclonal and polyclonal antibodies directed against the hCARM1 of the invention, and antibody fragments such as Fab′, sFv and F(ab′)2, can be used to block the action of the hCARM1 of the invention and allow study of the role of a particular hCARM1 of the invention. Alternatively, such antibodies can be used therapeutically to block the hCARM1 of the invention in a subject mammal, e.g., a human. The invention also includes a therapeutic composition comprising an antibody of the invention, and can also comprise a pharmaceutically acceptable carrier, solvent or diluent, and be administered by systems known in the art.

Antibodies that specifically bind to the hCARM1 of the invention, or fragments thereof, can also be used in immunohistochemical staining of tissue samples in order to evaluate the abundance and pattern expression of the hCARM1 of the invention. Antibodies can be used diagnostically in immunoprecipitation, immunoblotting, and enzyme linked immunosorbent assay (ELISA) to detect and evaluate levels of the hCARM1 of the invention in tissue or bodily fluid.

### EXAMPLES

### Example 1

### Expression Level of hCARM1-Long and hCARM1-Short

In order to determine the expression level of the two forms of CARM1 (Short form ═SF, Long Form=LF) RNA, specific primers (hCARM1-F1 (LF/SF): ATGCCGACCGCCTATGACT (SEQ ID NO:7); hCARM1-R1 (LF): GGAGGACCCTTGGACAATCC (SEQ ID NO:8); and hCARM1-RIB (SF): GGCGCCGGAGGACCCTAA (SEQ ID NO:9)) were designed for the performance of quantitative RT-PCR. The long and short forms used the same forward primer. As cDNA templates, an RNA collection derived from cell lines, tumor and normal tissue, and xenograft tumor material was used.

RNA quantification was performed using the Taqman® real-time-PCR fluorogenic assay, a precise method for assaying the concentration of nucleic acid templates.

All cell lines were grown using standard conditions: RPMI 1640 supplemented with 10% fetal bovine serum, 100 IU/ml penicillin, 100 mg/ml streptomycin, and 2 mM L-glutamine, 10 mM Hepes (all from GibcoBRL). Eighty percent confluent cells were washed twice with phosphate-buffered saline (GibcoBRL) and harvested using 0.25% trypsin (GibcoBRL). RNA was prepared using the RNeasy Maxi Kit from Qiagen. Tumor and normal tissue samples were bought from Ambion, Stratagene, Clontech, and Biochain. Xenograft tumor samples were harvested and prepared using the Rneasy Maxi Kit from Qiagen.

cDNA template for real-time PCR was generated using the Superscript™ First Strand Synthesis system for RT-PCR (Invitrogen).

SYBR Green real-time PCR reactions were prepared as follows: the reaction mix consisted of 20 ng first strand cDNA; 50 nM Forward Primer; 50 nM Reverse Primer; 0.75×SYBR Green I (Sigma); 1×SYBR Green PCR Buffer (50 mM Tris-HCl pH=8.3, 75 mM KCl); 10% DMSO; 3 mM MgCl2; 300 μM each dATP, dGTP, dTTP, dCTP; 1 U Platinum®8 Taq DNA Polymerase High Fidelity (Life Technologies Cat# 11304-029); 1:50 dil. ROX (Life Technologies). Real-time PCR was performed using an Applied Biosystems 5700 Sequence Detection System. Conditions were 95° C. for 10 min (denaturation and activation of Platinum® Taq DNA Polymerase), 40 cycles of PCR (95° C. for 15 sec, 60° C. for 1 min). PCR products are analyzed for uniform melting using an analysis algorithm built into the 5700 Sequence Detection System.

cDNA quantification used in the normalization of template quantity was performed using Taqman® technology. Taqman® reactions were prepared as follows: the reaction mix consisted of 20 ng first strand cDNA; 25 nM GAPDH-F3, Forward Primer; 250 nM GAPDH-R1 Reverse Primer; 200 nM GAPDH-PVIC Taqman® Probe (fluorescent dye labelled oligonucleotide primer); 1× Buffer A (Applied Biosystems); 5.5 mM MgCl2; 300 μM dATP, dGTP, dTTP, dCTP; 1 U Amplitaq Gold (Applied Biosystems). Real-time PCR was performed using an Applied Biosystems 7700 Sequence Detection System. Conditions were 95° C. for 10 min. (denaturation and activation of Amplitaq Gold), 40 cycles of PCR (95° C. for 15 sec, 60° C. for 1 min).

The sequences for the GAPDH oligonucleotides used in the Taqman® reactions were as follows:

| | |
|---|---|
| GAPDH-F3 - | |
| AGCCGAGCCACATCGCT; | (SEQ ID NO:10) |
| GAPDH-R1 - | |
| GTGACCAGGCGCCCAATAC; | (SEQ ID NO:11) |
| and | |
| GAPDH-PVIC Taqman ® Probe - | |
| VIC-CAAATCCGTTGACTCCGACCTTCACCTT- | (SEQ ID NO:12) |
| TAMRA. | |

The Sequence Detection System generates a Ct (threshold cycle) value that is used to calculate a concentration for each input cDNA template. cDNA levels for each gene of interest are normalized to GAPDH cDNA levels to compensate for variations in total cDNA quantity in the input sample. This is done by generating GAPDH Ct values for each cell line. Ct values for the gene of interest and GAPDH are inserted into the δδCt equation which is used to calculate a GAPDH normalized relative cDNA level for each specific cDNA.

Tissue sample RNA was obtained from Clinomics Biosciences, Inc. Total RNA was Dnase digested, purified using the RNAeasy Mini Kit from Qiagen and quality tested using Agilents Lab-on-a-Chip technique. 5 μg RNA were converted to cDNA using the Superscript™ First Strand Synthesis system for RT-PCR (Invitrogen).

SYBR Green real-time PCR reactions were prepared as it was the case for the other samples. However, in contrast to GAPDH normalization, the data were normalized to total input.

The tissue samples used are provided in Table 2.

TABLE 2
Tissue Samples

| Tissue | | Tissue | Tissue |
|---|---|---|---|
| Sample | Clinomics | Sample | Sample |
| Number | ID | Source | Description |
| 1 | M-0400 | Breast | Normal 44 year old female |
| 2 | M-0410 | Breast | Normal 53 year old female |
| 3 | M-0420 | Breast | Normal 31 year old female |
| 4 | M-0430 | Breast | Normal 42 year old female |
| 5 | M-0440 | Breast | Normal 66 year old female |
| 6 | M-0450 | Breast | Normal 73 year-old female |
| 7 | M-0460 | Breast | Normal 35 year old female |
| 8 | M-0470 | Breast | Normal 63 year old female |
| 9 | M-0100 | Breast | Adenocarcinoma, diagnostic type: DCIS, |
| | | | TNM staging: T1N0M0 |
| 10 | M-0110 | Breast | Adenocarcinoma, diagnostic type: DCIS, |
| | | | TNM staging: T1N0M0 |
| 11 | M-0111 | Breast | Adenocarcinoma, diagnostic type: IDC, |
| | | | TNM staging: T3N2M1 |
| 12 | M-0112 | Breast | Adenocarcinoma, diagnostic type: IDC, |
| | | | TNM staging: T3N2M2 |
| 13 | M-0113 | Breast | Adenocarcinoma, diagnostic type: IDC, |
| | | | TNM staging: T4N2M2 |
| 14 | M-0114 | Breast | Adenocarcinoma, diagnostic type: IDC, |
| | | | TNM staging: T4N2M1 |
| 15 | M-0115 | Breast | Adenocarcinoma, diagnostic type: IDC, |
| | | | TNM staging: T4N2M2 |
| 16 | M-0116 | Breast | Adenocarcinoma, diagnostic type: LC, |
| | | | TNM staging: T4N2M2 |
| 17 | M-0120 | Breast | Adenocarcinoma, diagnostic type: LCIS, |
| | | | TNM staging: T1N0M0 |
| 18 | M-0130 | Breast | Adenocarcinoma, diagnostic type: LCIS, |
| | | | TNM staging: T1N0M0 |
| 19 | M-0140 | Breast | Adenocarcinoma, diagnostic type: DCIS, |
| | | | TNM staging: T1N0M0 |
| 20 | M-0150 | Breast | Adenocarcinoma, diagnostic type: IDC, |
| | | | TNM staging: T1N0M0 |
| 21 | M-0160 | Breast | Adenocarcinoma, diagnostic type: IDC, |
| | | | TNM staging: T1N0M0 |
| 22 | M-0170 | Breast | Adenocarcinoma, diagnostic type: IDC, |
| | | | TNM staging: T2N0M0 |
| 23 | M-0180 | Breast | Adenocarcinoma, diagnostic type: IDC, |
| | | | TNM staging: T2N1M0 |
| 24 | M-0190 | Breast | Adenocarcinoma, diagnostic type: IDC, |
| | | | TNM staging: T2N1M0 |
| 25 | M-0600 | Colon | Normal 37year old male |
| 26 | M-0610 | Colon | Normal 35 year old female |
| 27 | M-0620 | Colon | Normal 53 year old male |
| 28 | M-0630 | Colon | Normal 35 year old female |
| 29 | M-0640 | Colon | Normal 31 year old female |
| 30 | M-0650 | Colon | Normal 44 year old male |
| 31 | M-0660 | Colon | Normal 63 year old female |
| 32 | M-0670 | Colon | Normal 44 year old male |
| 33 | M-0300 | Colon | Adenocarcinoma, Dukes stage: A, TNM |
| | | | staging: T1N0M0 |
| 34 | M-0310 | Colon | Adenocarcinoma, Dukes stage: A, TNM |
| | | | staging: T1N0M0 |
| 35 | M-0311 | Colon | Adenocarcinoma, Dukes stage: C, TNM |
| | | | staging: T2N2M0 |
| 36 | M-0312 | Colon | Adenocarcinoma, Dukes stage: C, TNM |
| | | | staging: T3N1M0 |
| 37 | M-0313 | Colon | Adenocarcinoma, Dukes stage: C, TNM |
| | | | staging: T3N2M1 |
| 38 | M-0314 | Colon | Adenocarcinoma, Dukes stage: D, TNM |
| | | | staging: T3N2M1 |
| 39 | M-0315 | Colon | Adenocarcinoma, Dukes stage: D, TNM |
| | | | staging: T3N2M2 |
| 40 | M-0316 | Colon | Adenocarcinoma, Dukes stage: D, TNM |
| | | | staging: T3N2M2 |
| 41 | M-0320 | Colon | Adenocarcinoma, Dukes stage: A, TNM |
| | | | staging: T1N0M0 |
| 42 | M-0330 | Colon | Adenocarcinoma, Dukes stage: A, TNM |
| | | | staging: T1N0M0 |
| 43 | M-0340 | Colon | Adenocarcinoma, Dukes stage: B, TNM |
| | | | staging: T1N0M0 |
| 44 | M-0350 | Colon | Adenocarcinoma, Dukes stage: B, TNM |
| | | | staging: T1N0M0 |
| 45 | M-0360 | Colon | Adenocarcinoma, Dukes stage: B, TNM |
| | | | staging: T2N0M0 |
| 46 | M-0370 | Colon | Adenocarcinoma, Dukes stage: B, TNM |
| | | | staging: T2N0M0 |
| 47 | M-0380 | Colon | Adenocarcinoma, Dukes stage: C, TNM |
| | | | staging: T2N2M0 |
| 48 | M-0390 | Colon | Adenocarcinoma, Dukes stage: C, TNM |
| | | | staging: T2N1M0 |
| 49 | M-0700 | Lung | Normal 56 Year old male |
| 50 | M-0710 | Lung | Normal 72 year old male |
| 51 | M-0720 | Lung | Normal 61 year old male |
| 52 | M-0730 | Lung | Normal 68 year old female |
| 53 | M-0740 | Lung | Normal 54 year old female |
| 54 | M-0750 | Lung | Normal 59 Year old female |
| 55 | T-400 | Lung | Normal, unknown donor |
| 56 | T-401 | Lung | Normal, unknown donor |
| 57 | M-0800 | Lung | Adenocarcinoma, cell type: Small Cell |
| 58 | M-0810 | Lung | Adenocarcinoma, cell type: Small Cell |
| 59 | M-0811 | Lung | Adenocarcinoma, |
| | | | cell type: Squamous Cell |
| 60 | M-0812 | Lung | Adenocarcinoma, |
| | | | cell type: Squamous Cell |
| 61 | M-0813 | Lung | Adenocarcinoma, |
| | | | cell type: Squamous Cell |
| 62 | M-0814 | Lung | Adenocarcinoma, |
| | | | cell type: Squamous Cell |
| 63 | M-0815 | Lung | Adenocarcinoma, |
| | | | cell type: Squamous Cell |
| 64 | M-0816 | Lung | Adenocarcinoma, |
| | | | cell type: Squamous Cell |
| 65 | M-0820 | Lung | Adenocarcinoma, |
| | | | cell type: Small Cell |
| 66 | M-0830 | Lung | Adenocarcinoma, |
| | | | cell type: Small Cell |
| 67 | M-0840 | Lung | Adenocarcinoma, |
| | | | cell type: Small Cell |
| 68 | M-0850 | Lung | Adenocarcinoma, |
| | | | cell type: Small Cell |
| 69 | M-0860 | Lung | Adenocarcinoma, |
| | | | cell type: Small Cell |
| 70 | M-0870 | Lung | Adenocarcinoma, |
| | | | cell type: Small Cell |
| 71 | M-0880 | Lung | Adenocarcinoma, |
| | | | cell type: Squamous Cell |
| 72 | M-0890 | Lung | Adenocarcinoma, |
| | | | cell type: Squamous Cell |
| 73 | M-0500 | Prostate | Normal 42 year old male |
| 74 | M-0510 | Prostate | Normal 53 year old male |
| 75 | M-0520 | Prostate | Normal 44 year old male |
| 76 | M-0530 | Prostate | Normal 44 year old male |
| 77 | M-0540 | Prostate | Normal 31 year old male |
| 78 | M-0550 | Prostate | Normal 63 year old male |
| 79 | M-0560 | Prostate | Normal 53 year old male |
| 80 | M-0570 | Prostate | Normal 63 year old male |
| 81 | M-0200 | Prostate | Adenocarcinoma, Gleason score: 3 |
| 82 | M-0210 | Prostate | Adenocarcinoma, Gleason score: 3 |
| 83 | M-0211 | Prostate | Adenocarcinoma, Gleason score: 9 |
| 84 | M-0212 | Prostate | Adenocarcinoma, Gleason score: 9 |
| 85 | M-0213 | Prostate | Adenocarcinoma, Gleason score: 9 |
| 86 | M-0214 | Prostate | Adenocarcinoma, Gleason score: 9 |
| 87 | M-0215 | Prostate | Adenocarcinoma, Gleason score: 9 |
| 88 | M-0216 | Prostate | Adenocarcinoma, Gleason score: 9 |
| 89 | M-0220 | Prostate | Adenocarcinoma, Gleason score: 4 |
| 90 | M-0230 | Prostate | Adenocarcinoma, Gleason score: 4 |
| 91 | M-0240 | Prostate | Adenocarcinoma, Gleason score: 5 |
| 92 | M-0250 | Prostate | Adenocarcinoma, Gleason score: 5 |
| 93 | M-0260 | Prostate | Adenocarcinoma, Gleason score: 7 |
| 94 | M-0270 | Prostate | Adenocarcinoma, Gleason score: 7 |
| 95 | M-0280 | Prostate | Adenocarcinoma, Gleason score: 7 |
| 96 | M-0290 | Prostate | Adenocarcinoma, Gleason score: 7 |

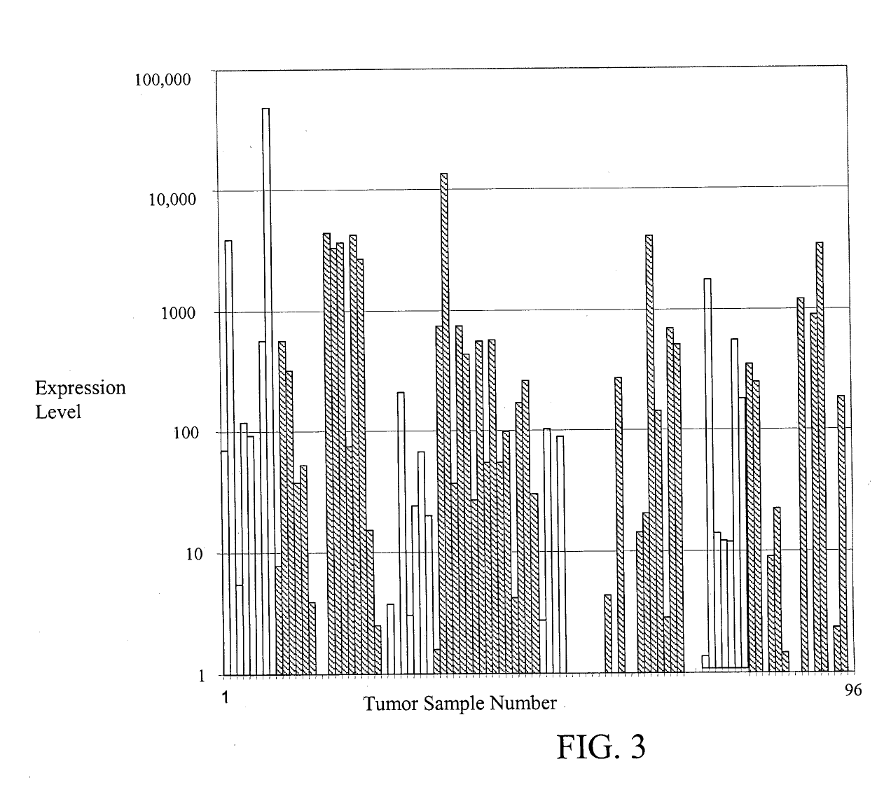

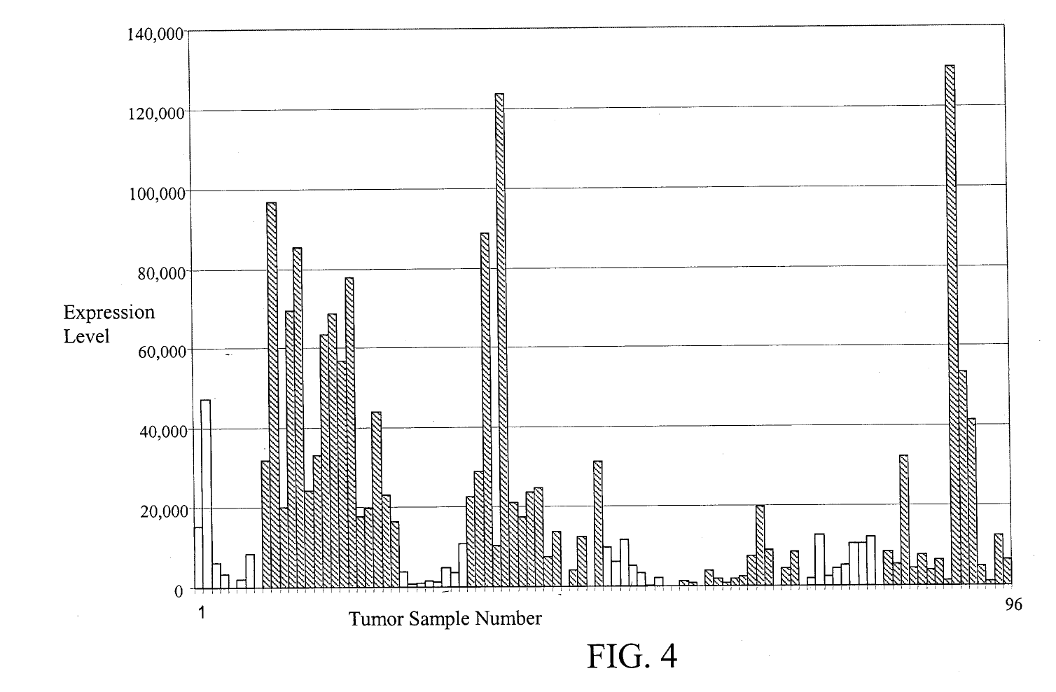

The resulting expression levels for hCARM1-long form and hCARM1-short form of the various tissue samples are provided in FIGS. 3 (hCARM1-long form) and 4 (hCARM1-short form), wherein each normal tissue sample is represented by an unpatterned bar and each tumor tissue sample is represented by a patterned bar. As shown in these figures, the hCARM1-short form had an expression level that was generally 2 to 40 fold higher than the hCARM1-long form, although there were some exceptions in some of the tissue samples.

### Example 2

### Methylation Assay

Methylation assay protocol: Reactions were performed in IX methylation buffer containing 20 mM Tris.HCl, pH 8.0, 200 mM NaCl and 0.4 mM EDTA. Reactions were assembled with 2.5 ug of Histone H3 and increasing amounts of hCARM1 (0.25 ug, 0.5 ug, 1.25 ug, 2.5 ug, 3.75 ug, 5 ug, or 7.5 ug). A mock reaction where hCARM1 was omitted was used as the negative control. Reactions were incubated at 30° C. for 1 hr. prior to loading on a 10-20% gradient SDS-PAGE. The gel was fixed, dried and exposed to film.

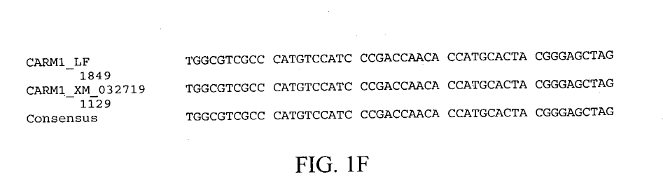

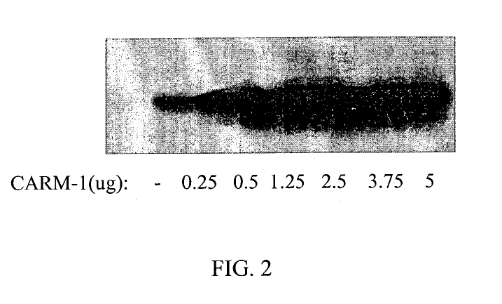

A methylation reaction was performed in order to evaluate whether the cloned full-length hCARM1 had methylating activity. Mouse CARM1 has been previously shown to specifically methylate Histone H3 in vitro and in vivo. Experiments were conducted to determine whether the human homolog was also capable of exhibiting the same substrate preference. hCARM1 was produced in and purified from baculovirus infected insect cells and increasing amounts of the purified enzyme were added to reactions containing a constant amount of recombinant Histone H3. The results demonstrated that hCARM1 methylates Histone H3 efficiently (FIG. 2). Interestingly, a previously documented general methylation inhibitor homocysteine effectively inhibited hCARM1 mediated methylation.

### Example 3

### Assay for High Through-Put Screening for Inhibitors of CARM1 Enzymatic Activity

A scintillation proximity assay (SPA) based on the enzymatic activity of CARM1 was devised to screen for compounds that specifically inhibited CARM1 dependent methylation. Human full-length CARM1 purified from baculovirus-infected insect cells was used as the source for enzyme. Histone H3 (Roche Applied Science) was used as the substrate for the assay since methylation of CARM1 on several arginine residues in the N- and C-terminal tails of Histone H3 has been well-documented. Tritiated S-Adenosyl-L-Methionine (SAM) (Amersham Pharmacia Biotech) was used as a cofactor since the methylating activity of CARM1 exhibits an absolute requirement for SAM. The reaction was allowed to proceed at room temperature for two hours in the presence of methylation buffer (20 mM Tris. HCl. pH 8.0, 200 mM NaCl, 0.4 mM EDTA) and presence or absence of compound. The reaction was stopped using 0.1N HCl and the methylated Histone H3 captured by an antibody (Upstate Biotechnology) that specifically recognizes the methylated arginine 17 residue in the N-terminus of Histone H3. The antibody was previously bound to polystyrene Lead Seeker beads coated with Protein A (Amersham Pharmacia Biotech). Beads were allowed to settle for 6 hr. before the plates were counted in a Lead Seeker imaging system (Amersham Pharmacia Biotech).

### Example 4

### Cell-Based Assays

Transfection protocol: Cells were plated in 12 well-dishes and allowed to adhere and grow overnight such that they were 80% confluent at the time of transfection. Tranfections were performed in triplicate using Lipofectamine 2000 (Gibco) and OptiMEM media. Total amount of DNA transfected was held constant within experiments. Six hrs. post transfection the Lipofectamine-DNA mix was removed and replaced with fresh media containing 10% serum. Hormone (dihydrotestosterone or estradiol) was added at this time and reporter activation measured after 24 hr.

Mouse CARM1 has been implicated as a coactivator of the androgen and estrogen receptor mediated signaling pathways along with the well-known steroid coactivator GRIP-1. The contribution, if any, of the human clone to these pathways was investigated. When full-length hCARM1 was co-transfected with GRIP-1 and the estrogen receptor (ER) into the breast cancer cell-line T47D, a clear hCARM1 concentration dependent increase in the estradiol mediated induction of a reporter construct containing an ER dependent promoter in front of the luciferase gene was obtained when compared to the induction obtained with GRIP-1 and ER alone. Conversely, co-transfection of antisense oligos to hCARM1 effectively abrogated activation of the ER dependent reporter in the presence of transfected hCARM1. Interestingly, a similar inhibitory effect on ER dependent activation could be obtained by transfection of CARM1 antisense oligos or short interfering RNAs (siRNAs) even in the absence of any exogenous CARM1 protein. Thus, antagonizing endogenous CARM1 is deleterious to hormone dependent activation by endogeous ER. Similar results were obtained upon cotransfection of hCARM1 antisense oligos into MDA-MB-453 breast cancer cells to assess androgen receptor (AR) dependent signaling. These results implicate an essential role for hCARM1 in AR and ER mediated signaling in cells.

Although the invention has been described in some detail by way of illustration and example for purposes of clarity and understanding, it will be apparent that certain changes and modifications may be practiced within the scope of the appended claims.

1. An isolated polynucleotide comprising:
(a) a nucleotide sequence encoding a coactivator-associated arginine methyltransferase 1 polypeptide wherein the amino acid sequence of the polypeptide and the amino acid sequence of SEQ ID NO:4 have at least 95% sequence identity; or
(b) the complement of the nucleotide sequence, wherein the complement and the nucleotide sequence contain the same number of nucleotides and are 100% complementary.

2. The polynucleotide of claim 1 wherein the polynucleotide encodes the polypeptide of SEQ ID NO:4.

3. The polynucleotide of claim 1 that comprises the nucleotide sequence of SEQ ID NO:3.

4. An isolated polynucleotide comprising:
(a) a nucleotide sequence encoding a coactivator-associated arginine methyltransferase 1 polypeptide wherein the amino acid sequence of the polypeptide and the amino acid sequence of SEQ ID NO:6 have at least 95% sequence identity; or
(b) the complement of the nucleotide sequence, wherein the complement and the nucleotide sequence contain the same number of nucleotides and are 100% complementary.

5. The polynucleotide of claim 4 wherein the polynucleotide encodes the polypeptide of SEQ ID NO:6.

6. The polynucleotide of claim 4 that comprises the nucleotide sequence of SEQ ID NO:5.

7. An expression vector comprising the polynucleotide of claim 1 and an expression control sequence operatively linked to the polynucleotide.

8. A process for producing a recombinant host cell comprising transforming or transfecting a host cell with the expression vector of claim 7 such that the host cell, under appropriate culture conditions, produces a coactivator-associated arginine methyltransferase 1 polypeptide.

9. A recombinant host cell produced by the process of claim 8.

10. An isolated coactivator-associated arginine methyltransferase 1 polypeptide comprising an amino acid sequence that has at least 95% sequence identity to the amino acid sequence of SEQ ID NO:4.

11. The polypeptide of claim 10 that comprises the amino acid sequence of SEQ ID NO:4.

12. An isolated coactivator-associated arginine methyltransferase 1 polypeptide comprising an amino acid sequence that has at least 95% sequence identity to the amino acid sequence of SEQ ID NO:6.

13. The polypeptide of claim 12 that comprises the amino acid sequence of SEQ ID NO:6.

14. A process for producing a coactivator-associated arginine methyltransferase 1 polypeptide comprising culturing the recombinant host cell of claim 9 under conditions sufficient for the production of said polypeptide and recovering the polypeptide.

15. A method for identifying a substance which is capable of modulating a coactivator-associated arginine methyltransferase 1 molecule or a fragment thereof, said method comprising the steps of:
(a) reacting the coactivator-associated arginine methyltransferase 1 polypeptide of claim 10 with a candidate substance under conditions which permit an interaction between said coactivator-associated arginine methyltransferase 1 polypeptide and said candidate substance; and
(b) assaying for one or more of a candidate substance-coactivator-associated arginine methyltransferase 1 polypeptide complex, a free coactivator-associated arginine methyltransferase 1 polypeptide, a non-complexed candidate substance, or activation of the coactivator-associated arginine methyltransferase 1 polypeptide.

16. A method for identifying a substance which is capable of modulating a coactivator-associated arginine methyltransferase 1 molecule or a fragment thereof, said method comprising the steps of:
(a) reacting the coactivator-associated arginine methyltransferase 1 polypeptide of claim 12 with a candidate substance under conditions which permit an interaction between said coactivator-associated arginine methyltransferase 1 polypeptide and said candidate substance; and
(b) assaying for one or more of a candidate substance-coactivator-associated arginine methyltransferase 1 polypeptide complex, a free coactivator-associated arginine methyltransferase 1 polypeptide, a non-complexed candidate substance, or activation of the coactivator-associated arginine methyltransferase 1 polypeptide.


SEQUENCE LISTING (12 sequences)

SEQ ID NO: 1 - protein, 35 aa, Homo sapiens
CACCGAATTCGCCGGATCTAAGATGGCAGCGGCGG

SEQ ID NO: 2 - DNA, 30 bp, Homo sapiens
ctagctcccgtagtgcatggtgttggtcgg

SEQ ID NO: 3 - DNA, 1780 bp, Homo sapiens
caccgaattcgccggatctaagatggcagcggcggcggcggcggtggggccgggcgcgggcggcgcggggtcggcggtcccgggcggcgcggggccctgcgctaccgtgtcggtgttccccggcgcccgcctcctcaccatcggcgacgcgaacggcgagatccagcggcacgcggagcagcaggcgctgcgcctcgaggtgcgcgccggcccggactcggcgggcatcgccctctacagccatgaagatgtgtgtgtctttaagtgctcagtgtcccgagagacagagtgcagccgtgtgggcaagcagtccttcatcatcaccctgggctgcaacagcgtcctcatccagttcgccacacccaacgatttctgttccttctacaacatcctgaaaacctgccggggccacaccctggagcggtctgtgttcagcgagcggacggaggagtcttctgccgtgcagtacttccagttttatggctacctgtcccagcagcagaacatgatgcaggactacgtgcggacaggcacctaccagcgcgccatcctgcaaaaccacaccgacttcaaggacaagatcgttcttgatgttggctgtggctctgggatcctgtcgttttttgccgcccaagctggagcacggaaaatctacgcggtggaggccagcaccatggcccagcacgctgaggtcttggtgaagagtaacaacctgacggaccgcatcgtggtcatcccgggcaaggtggaggaggtgtcactccccgagcaggtggacatcatcatctcggagcccatgggctacatgctcttcaacgagcgcatgctggagagctacctccacgccaagaagtacctgaagcccagcggaaacatgtttcctaccattggtgacgtccaccttgcacccttcacggatgaacagctctacatggagcagttcaccaaggccaacttctggtaccagccatctttccatggagtggacctgtcggccctccgaggtgccgcggtggatgagtatttccggcagcctgtggtggacacatttgacatccggatcctgatggccaagtctgtcaagtacacggtgaacttcttagaagccaaagaaggagatttgcacaggatagaaatcccattcaaattccacatgctgcattcagggctggtccacggcctggctttctggtttgacgttgctttcatcggctccataatgaccgtgtggctgtccacagccccgacagagcccctgacccactggtaccaggtgcggtgcctgttccagtcaccactgttcgccaaggcaggggacacgctctcagggacatgtctgcttattgccaacaaaagacagagctacgacatcagtattgtggcccaggtggaccagaccggctccaagtccagtaacctcctggatctgaaaaaccccttctttagatacacgggcacaacgccctcacccccacccggctcccactacacatctccctcggaaaacatgtggaacacgggcagcacctacaacctcagcagcgggatggccgtggcagggatgccgaccgcctatgacttgagcagtgttattgccagtggctccagcgtgggccacaacaacctgattcctttagggtcctccggcgcccagggcagtggtggtggcagcacgagtgcccactatgcagtcaacagccagttcaccatgggcggccccgccatctccatggcgtcgcccatgtccatcccgaccaacaccatgcactacgggagctag

SEQ ID NO: 4 - protein, 585 aa, Homo sapiens
MAAAAAAVGPGAGGAGSAVPGGAGPCATVSVFPGARLLTIGDANGEIQRHAEQQALRLEVRAGPDSAGIALYSHEDVCVFKCSVSRETECSRVGKQSFIITLGCNSVLIQFATPNDFCSFYNILKTCRGHTLERSVFSERTEESSAVQYFQFYGYLSQQQNMMQDYVRTGTYQRAILQNHTDFKDKIVLDVGCGSGILSFFAAQAGARKIYAVEASTMAQHAEVLVKSNNLTDRIVVIPGKVEEVSLPEQVDIIISEPMGYMLFNERMLESYLHAKKYLKPSGNMFPTIGDVHLAPFTDEQLYMEQFTKANFWYQPSFHGVDLSALRGAAVDEYFRQPVVDTFDIRILMAKSVKYTVNFLEAKEGDLHRIEIPFKFHMLHSGLVHGLAFWFDVAFIGSIMTVWLSTAPTEPLTHWYQVRCLFQSPLFAKAGDTLSGTCLLIANKRQSYDISIVAQVDQTGSKSSNLLDLKNPFFRYTGTTPSPPPGSHYTSPSENMWNTGSTYNLSSGMAVAGMPTAYDLSSVIASGSSVGHNNLIPLGSSGAQGSGGGSTSAHYAVNSQFTMGGPAISMASPMSIPTNTMHYGS

SEQ ID NO: 5 - DNA, 1849 bp, Homo sapiens
caccgaattcgccggatctaagatggcagcggcggcggcggcggtggggccgggcgcgggcggcgcggggtcggcggtcccgggcggcgcggggccctgcgctaccgtgtcggtgttccccggcgcccgcctcctcaccatcggcgacgcgaacggcgagatccagcggcacgcggagcagcaggcgctgcgcctcgaggtgcgcgccggcccggactcggcgggcatcgccctctacagccatgaagatgtgtgtgtctttaagtgctcagtgtcccgagagacagagtgcagccgtgtgggcaagcagtccttcatcatcaccctgggctgcaacagcgtcctcatccagttcgccacacccaacgatttctgttccttctacaacatcctgaaaacctgccggggccacaccctggagcggtctgtgttcagcgagcggacggaggagtcttctgccgtgcagtacttccagttttatggctacctgtcccagcagcagaacatgatgcaggactacgtgcggacaggcacctaccagcgcgccatcctgcaaaaccacaccgacttcaaggacaagatcgttcttgatgttggctgtggctctgggatcctgtcgttttttgccgcccaagctggagcacggaaaatctacgcggtggaggccagcaccatggcccagcacgctgaggtcttggtgaagagtaacaacctgacggaccgcatcgtggtcatcccgggcaaggtggaggaggtgtcactccccgagcaggtggacatcatcatctcggagcccatgggctacatgctcttcaacgagcgcatgctggagagctacctccacgccaagaagtacctgaagcccagcggaaacatgtttcctaccattggtgacgtccaccttgcacccttcacggatgaacagctctacatggagcagttcaccaaggccaacttctggtaccagccatctttccatggagtggacctgtcggccctccgaggtgccgcggtggatgagtatttccggcagcctgtggtggacacatttgacatccggatcctgatggccaagtctgtcaagtacacggtgaacttcttagaagccaaagaaggagatttgcacaggatagaaatcccattcaaattccacatgctgcattcagggctggtccacggcctggctttctggtttgacgttgctttcatcggctccataatgaccgtgtggctgtccacagccccgacagagcccctgacccactggtaccaggtgcggtgcctgttccagtcaccactgttcgccaaggcaggggacacgctctcagggacatgtctgcttattgccaacaaaagacagagctacgacatcagtattgtggcccaggtggaccagaccggctccaagtccagtaacctcctggatctgaaaaaccccttctttagatacacgggcacaacgccctcacccccacccggctcccactacacatctccctcggaaaacatgtggaacacgggcagcacctacaacctcagcagcgggatggccgtggcagggatgccgaccgcctatgacttgagcagtgttattgccagtggctccagcgtgggccacaacaacctgattcctttagccaacacggggattgtcaatcacacccactcccggatgggctccataatgagcacggggattgtccaagggtcctccggcgcccagggcagtggtggtggcagcacgagtgcccactatgcagtcaacagccagttcaccatgggcggccccgccatctccatggcgtcgcccatgtccatcccgaccaacaccatgcactacgggagctag

SEQ ID NO: 6 - protein, 608 aa, Homo sapiens
MAAAAAAVGPGAGGAGSAVPGGAGPCATVSVFPGARLLTIGDANGEIQRHAEQQALRLEVRAGPDSAGIALYSHEDVCVFKCSVSRETECSRVGKQSFIITLGCNSVLIQFATPNDFCSFYNILKTCRGHTLERSVFSERTEESSAVQYFQFYGYLSQQQNMMQDYVRTGTYQRAILQNHTDFKDKIVLDVGCGSGILSFFAAQAGARKIYAVEASTMAQHAEVLVKSNNLTDRIVVIPGKVEEVSLPEQVDIIISEPMGYMLFNERMLESYLHAKKYLKPSGNMFPTIGDVHLAPFTDEQLYMEQFTKANFWYQPSFHGVDLSALRGAAVDEYFRQPVVDTFDIRILMAKSVKYTVNFLEAKEGDLHRIEIPFKFHMLHSGLVHGLAFWFDVAFIGSIMTVWLSTAPTEPLTHWYQVRCLFQSPLFAKAGDTLSGTCLLIANKRQSYDISIVAQVDQTGSKSSNLLDLKNPFFRYTGTTPSPPPGSHYTSPSENMWNTGSTYNLSSGMAVAGMPTAYDLSSVIASGSSVGHNNLIPLANTGIVNHTHSRMGSIMSTGIVQGSSGAQGSGGGSTSAHYAVNSQFTMGGPAISMASPMSIPTNTMHYGS

SEQ ID NO: 7 - DNA, 19 bp, Homo sapiens
atgccgaccgcctatgact

SEQ ID NO: 8 - DNA, 20 bp, Homo sapiens
ggaggacccttggacaatcc

SEQ ID NO: 9 - DNA, 18 bp, Homo sapiens
ggcgccggaggaccctaa

SEQ ID NO: 10 - DNA, 17 bp, Homo sapiens
agccgagccacatcgct

SEQ ID NO: 11 - DNA, 19 bp, Homo sapiens
gtgaccaggcgcccaatac

SEQ ID NO: 12 - DNA, 28 bp, Homo sapiens
caaatccgttgactccgaccttcacctt